(19) United States
(12) Patent Application Publication
(10) Pub. No.: US 2020/0046638 A1
(43) Pub. Date: Feb. 13, 2020

(54) PHARMACEUTICAL COMPOSITION FOR THE TREATMENT OF SINUSITIS AND COPD

(71) Applicant: COMBISIN SÀRL, Lausanne (CH)

(72) Inventor: Matthew KRAYENBUHL, Pully (CH)

(21) Appl. No.: 16/340,520

(22) PCT Filed: Oct. 10, 2017
(86) PCT No.: PCT/IB2017/056250

§ 371 Date: Apr. 9, 2019

(30) Foreign Application Priority Data
Oct. 10, 2016 (IB) PCT/IB2016/056058

(51) Int. Cl. A61K 9/00; A61K 45/06; A61K 47/10; A61K 31/573; A61K 31/58; A61K 31/14

(52) U.S. Cl. CPC A61K 9/0073; A61K 45/06; A61K 9/0043; A61K 31/14; A61K 31/573; A61K 31/58; A61K 47/10

(57) ABSTRACT

The invention relates to a Pharmaceutical composition of a puff of a spray or inhaler for use in the treatment of sinusitis or/and COPD, by administration by inhalation to a human subject comprising at least one topical corticoid, a sugar alcohol, and an antiseptic wherein said topical corticoid is present in an amount between 10 and 1000 μg, the sugar alcohol being present in a percentage between 0.1 and 20, and the antiseptic present in a percentage between 0.001 and 5. This composition can be administered by nasal spray or dry powder inhaler and is used for treating the upper and lower respiratory tract, in particular sinusitis with or without polyps, as well as COPD or other respiratory deceases as mucoviscidosis.

### FIELD OF THE INVENTION

The present invention relates to pharmaceutical compositions for the treatment of sinusitis and chronic obstructive pulmonary disease (COPD) that comprise a topical corticoid

### PRIOR ART

Compositions such as those described above are disclosed in international patent application WO 2012/127407 that designate the same inventor as the present application.

### GENERAL DESCRIPTION OF THE INVENTION

The problem that the present invention aims to solve is to provide alternatives and/or improvements of the treatments currently offered, in particular treatments for upper and lower respiratory tracts, namely sinusitis and/or COPD. Improvement means “increased efficacy of the treatment and/or decrease in toxicity and/or minimization of the amounts used (e.g. of antibiotic)”.

In the invention, the solution of the aforementioned problem consists of a pharmaceutical composition of a puff of a spray for use in the treatment of sinusitis and/or COPD, by inhalation by a human subject comprising at least one topical corticoid, a sugar alcohol and an antiseptic wherein said topical corticoid is present in an amount between 10 and 1000 μg and said sugar alcohol being present in a percentage between 0.1 and 20 and said antiseptic, preferably, benzalkonium chloride, in a percentage between 0.001 and 5.

A unit dose is the amount of a pharmaceutical composition administered to a patient in a single dose, such as a puff of spray.

The pharmaceutical composition of unit dose according to the invention comprises a first active ingredient selected from the topical corticoids. These corticoids (also called steroids, corticosteroids, glucocorticoids or cortisone analogues) are compounds that are generally used for local application (nasal, cutaneous, ophthalmic, etc.). Examples of these topical corticoids include: mometasone furoate, budesonide, beclometasone, fluticasone furoate, fluticasone propionate, triamcinolone.

The preferred corticoid is budenoside and triamcinolone.

The pharmaceutical composition of unit dose according to the invention further comprises a second active ingredient that is a sugar alcohol and a third active ingredient which is an antiseptic, quaternary ammoniums, for example.

Sugar alcohols have the general formula HOCH₂ (CHOH)ₙ CH₂ OH

Any suitable sugar alcohol can be used in the composition according to the invention.

Any antiseptic can be used in the composition according to the invention, preferably quaternary ammoniums.

In one embodiment the composition has no antibiotics.

In another embodiment the composition comprises an antibiotic but other than quinolone or fucidic acid.

The pharmaceutical composition of unit dose according to the invention can also comprise one or more pharmaceutically acceptable excipients. These excipients can be selected from dispersants, solubilizers, stabilizers, preservatives, antioxidants, etc.

The pharmaceutical composition of unit dose according to the invention is advantageously formulated as a liquid (solution, suspension, emulsion, etc.) or dry powder, in unit dose or in a multi-dose bottle for administration by spray in to the nasal cavity and the sinuses for treatment of the respiratory passages, and as dry powder or liquid to the lungs. This composition includes a powder that can be mixed with a diluent to produce a liquid.

The composition of unit dose according to the invention is used in the treatment of the upper and lower respiratory tracts, in the treatment of sinusitis, and COPD, in particular of acute or/and chronic sinusitis nasal polyps and delivered to the nose through a metered spray with volumes ranging from 20 μl to 400 μl, most preferred 100 μl. In the case of a lungs/bronchi treatment or COPD, the inhaler will deliver 20 μl to 800 μl.

The invention also relates to a pharmaceutical preparation containing several puffs of spray according to the invention and being in the form of nasal spray or lungs/bronchi inhaler.

### EXAMPLE 1

Positive results have been observed with a pharmaceutical composition of unit dose comprising triamcinolone (40 μg), mannitol (2%) and benzalkonium (0.006%). The dose was administrated by nasal spray.

### EXAMPLE 2

Other positive results have been observed with a pharmaceutical composition of unit dose, also administrated by nasal spray, that comprises budesonide (50 μg), mannitol (2%) and benzalkonium (0.006%).

1. Pharmaceutical composition of a puff of a spray for use in the treatment of sinusitis and/or COPD by administration by inhalation to a human subject comprising at least one topical corticoid, a sugar alcohol and an antiseptic, wherein said topical corticoid is present in an amount between 10 and 1000 μg, the said sugar alcohol being present in a percentage between 0.1 and 20 and the antiseptic in a percentage between 0.001 and 5.

2. Pharmaceutical composition of unit dose according to claim 1 wherein said antiseptic is benzalkonium chloride.

3. Pharmaceutical composition of unit dose according to claim 1 in which the topical corticoid is selected from the group of the following molecules: mometasone furoate, budesonide, beclometasone, fluticasone furoate, fluticasone propionate, triamcinolone.

4. Pharmaceutical composition according to claim 1 that contains no antibiotics.

5. Pharmaceutical composition according to claim 1 furthermore comprising an antibiotics other than quinolone or fucidic acid.

6. Pharmaceutical preparation containing several puffs of a spray according to claim 1, wherein it is in the form of nasal spray or lungs/bronchi inhaler.
